(19) United States
(12) Patent Application Publication
(10) Pub. No.: US 2007/0038192 A1
(43) Pub. Date: Feb. 15, 2007

(54) Hygiene article

(75) Inventor: Martin Bentz, Heidenheim (DE)

Correspondence Address: HOLLAND & KNIGHT LLP;ATTN: STEFAN V. STEIN/ IP DEPT., POST OFFICE BOX 1288, TAMPA, FL, 33601-1288, US

(73) Assignee: Outlast Technologies

(21) Appl. No.: 11/481,175

(22) Filed: Jul. 5, 2006

(30) Foreign Application Priority Data
Jul. 14, 2005 (DE) 10 2005 032 769.9

(51) Int. Cl. A61M 1/00

(52) U.S. Cl. 604/317

(57) ABSTRACT

Hygiene articles, such as a sanitary napkin, a tampon or an incontinence pad, are formed from a textile fabric having a multiplicity of fibres. Microcapsules having reversible heat storage properties are applied in at least a portion of the fibres or via a coating with a binder atop at least a portion of the fibres or atop the surfaces of the fabric. The microcapsules include materials in which a phase transition from solid to liquid, and vice versa, takes place in a temperature range from 15 to 40° C.

The invention relates to a hygiene article, such as a sanitary napkin, a tampon or an incontinence pad, consisting of a textile fabric formed by a multiplicity of fibres.

The invention also relates to a process for producing a hygiene article.

Hygiene articles usually utilize fibres which possess high absorbency by virtue of their foundation material and their cross section. Viscose is frequently utilized as foundation material. The fibres are generally processed to form flexible, porous textile fabrics, usually fibrous nonwoven web fabrics, not by interweaving of warp and weft or by interlooping, but by interentanglement and/or cohesive and/or adhesive interbonding of textile fibres. This produces looser fabrics, which are very absorbent. Known fibres for making the fibrous nonwoven webs are manufactured fibres or filaments, usually of polypropylene, polyester or viscose, their coherency generally being due to their autoadhesion. Adhesively consolidated fibrous nonwoven webs are formed by interadhering the fibres with liquid binders or by melting or dissolving binder fibres admixed to the fibrous nonwoven web in the course of its production.

In cohesive consolidation, fibre surfaces are solubilized by. suitable chemicals and conjoined by pressure or welded together at elevated temperature.

The disadvantage with existing hygiene articles is that temperature elevations, for example in the event of high ambient temperatures or physical activities on the part of the person using the hygiene article, give rise to perspiration or sweating complete with the problems resulting therefrom.

It is an object of the present invention to provide a hygiene article of the kind mentioned at the beginning that offers a higher wear comfort, more particularly is capable of accommodating excess body heat and hence of reducing perspiration or sweating.

In accordance with the invention, this object is achieved when microcapsules having reversible heat-storing properties are included in at least a portion of the fibres or are applied via a coating with a binder atop at least a portion of the fibres or of the surface of the fabric, the microcapsules including materials in which a phase transition from solid to liquid, and vice versa, takes place in a temperature range from 15 to 40° C.

The invention's incorporation of fibres comprising microcapsules comprising phase transition materials provides distinctly improved wear comfort to the fabric. Excess body heat can be accommodated by the phase transition materials in the microcapsules whereby overheating of the body of the person wearing or using the hygiene article, and hence perspiration or sweating, can be distinctly reduced. The invention's fibres with microcapsules are able to smooth out temperature fluctuations of the body for a prolonged period. Dynamic temperature regulation is achieved in this way.

The hygiene article of the invention utilizes a law of nature. At a phase change temperature, in which material changes from solid to liquid or vice versa, the material absorbs and holds a large quantity of thermal energy at a constant temperature before changing to the next phase. Thus, the material can be used as an absorber, for example for a hygiene article, in which the microcapsules are integrated or otherwise arranged, before its temperature can rise. Conversely, it is likewise possible to use an appropriately preheated material as a barrier to cold, since, for a transition from liquid to solid, thermal energy must first be removed from the phase change material before its temperature can drop. However, the last is less interesting for hygiene articles.

The hygiene article of the invention can be produced in various ways.

One possible example is for fibres comprising the invention's microcapsules in which the phase transition materials are included to be produced separately and these fibres then to be combined with other fibres for processing to form a fibrous nonwoven web. It is also possible of course for the entire fibrous nonwoven web to be produced from the invention's fibres comprising microcapsules and phase transition materials.

Alternatively it is also possible for hitherto used “normal” fibres or yarns to be used, in which case these are then provided with a coating with a binder before their processing to form a textile fabric, for example a fibrous nonwoven web, in which case microcapsules with the phase transition materials of the invention are then dispersed or otherwise accommodated in the binder.

Instead of a coating of individual fibres, it is also possible for the textile fabric produced from the “standard” fibres correspondingly to be provided with a coating with a binder and, included in the binder, microcapsules comprising phase transition materials.

The coating may be effected for example with a liquid polymer binder, which is applied in a dipping or spraying operation. Of course, a printing, coating or spreading process is also possible.

The textile fabrics which, in accordance with the present invention, are provided with the microcapsules and the phase transition materials included in the microcapsules can then be further processed using known manufacturing sequences to form the desired end product in a conventional manner.

An illustrative embodiment of the invention will now be described in principle with reference to the drawing, where

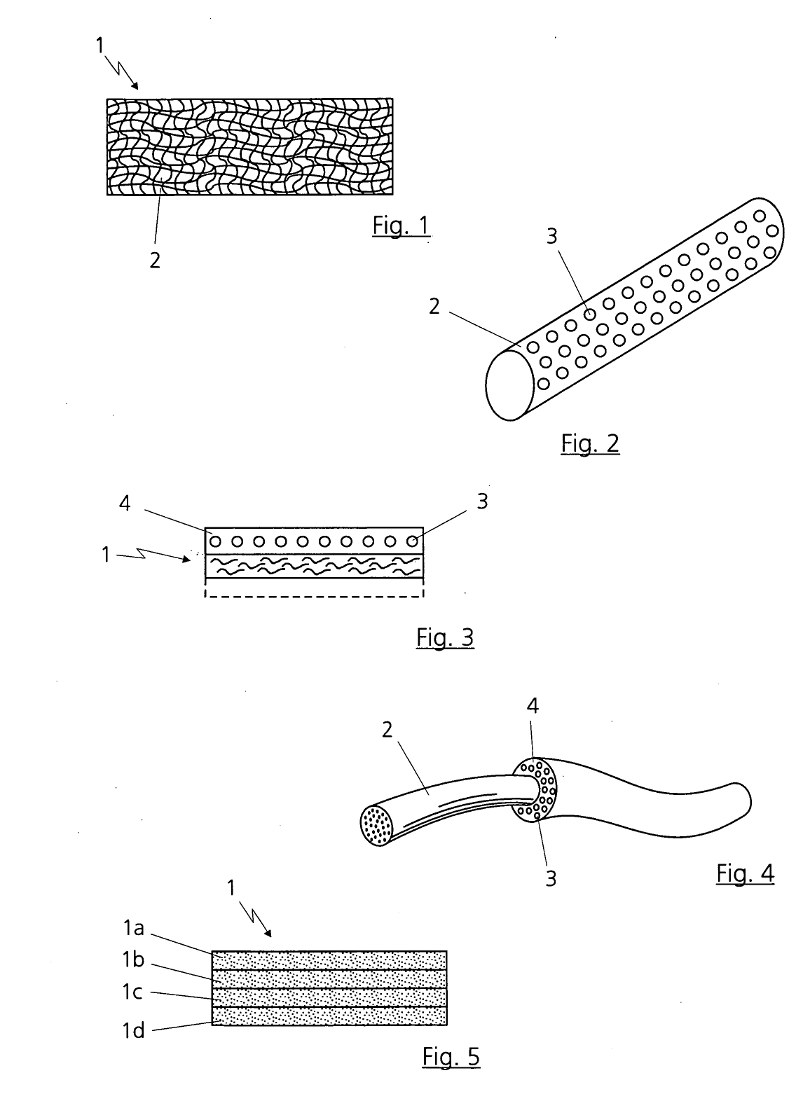

FIG. 1 shows, as hygiene article, a sanitary napkin in plan view;

FIG. 2 shows a greatly magnified presentation of a fibre comprising microcapsules;

FIG. 3 shows an enlarged detail of a sanitary napkin comprising a coating comprising a binder and microcapsules;

FIG. 4 shows a magnified representation of a fibre provided with a coating from a binder comprising microcapsules; and

FIG. 5 shows a hygiene article consisting of a plurality of plies of textile fabrics.

The sanitary napkin presented in FIG. 1 consists of a textile fabric 1 in the form of a fibrous nonwoven web comprising a multiplicity of fibres 2. Instead of the sanitary napkin presented in FIG. 1, tampons, incontinence pads, diapers and the like are also possible as hygiene article.

The hygiene article 1 is provided with a multiplicity of microcapsules 3 incorporated in the textile fabric in the manner described hereinbelow.

FIG. 2 shows in a greatly magnified perspective presentation a fibre 2 in which a multiplicity of microcapsules 3 are integrated. The foundation material for the fibre 2 can be for example polymer or else cellulose. The microcapsules 3 include one or more materials in which the phase transition from solid to liquid, and vice versa, takes place in a temperature range from 15 to 40° C. and preferably in the range from 18 to 35° C. A wide variety of materials are suitable for this, which are used either individually or in mixtures, for example in order to be able to cover, by means of different transition temperatures, a broader temperature spectrum within which heat storage by the phase transition materials takes place in the course of their transition from solid to liquid.

Such microcapsules and phase transition materials as well as integration in fibres are already known in principle. See for example EP 0 306 202 B1 (=U.S. Pat. No. 4,756,958), the disclosure content of which is also fully part of the present invention. Examples of thermal transition materials useful in the interior of the microcapsules 3 are paraffinic, phase-changing hydrocarbonates or else plastically deformable crystalline materials.

Useful crystalline materials include for example 2,2-dimethyl-1,3-propanediol (DMP) and 2-hydrooxymethyl-2-methyl-1,3-propanediol (HMP). When such crystals absorb thermal energy, the molecular structure is temporarily modified without changing the phase of the material.

The foundation material for the fibre can be a synthetic polymer, for example polyester, nylon, acrylic or modacrylic or a cellulose fibre, for example viscose.

The size of the microcapsules 3 can be between 0.5 μm and 1000 μm, preferably between 2 and 30 μm.

The textile fabric 1 of FIG. 1 for the hygiene article can be produced not only exclusively from the FIG. 2 fibre 2 comprising the microcapsules 3 but also in conjunction with standard fibres, and in the latter case the fibres 2 comprising the microcapsules 3 are appropriately combined to form a composite with the other standard fibres.

FIG. 3 depicts a textile fabric 1 provided with a coating 4 in the form of liquid polymer binder in which a multiplicity of microcapsules 3 have been dispersed. The coating may be provided on just one side or else on both sides (see broken-line representation).

The coating process and its construction are already known in principle, which is why no further details will be provided here. See for example EP 0 611 330 B1 (=U.S. Pat. No. 5,366,801), which likewise forms part of the disclosure content of the present invention.

Instead of coating the textile fabric 1, it is also possible, as depicted in FIG. 4, to envelop standard fibres 2 individually with the coating 4 in which the microcapsules 3 have been dispersed, and then to produce a fibrous nonwoven web therefrom or in conjunction with other fibres.

Instead of a production of the hygiene article from a fibrous nonwoven web comprising a multiplicity of individual fibres 2, the hygiene article can also be produced from multicomponent fibres comprising microcapsules. The textile fabric 1 can be formed from multicomponent fibres as a fibre body, in which case at least a portion of the fibres includes microcapsules 3. This process is likewise already known in principle and shall for its disclosure content likewise form part of the content of the present invention. Reference is made to US 2003/0035951 A1.

It is similarly possible to produce the hygiene article from cellulosic fibres comprising microcapsules 3. WO 2005/017247 A2 supplies further details, which shall likewise form part of the disclosure content of the present invention. The advantage of cellulosic fibres is that they are able to take up appreciable amounts of moisture. However, conventional cellulosic fibres are disadvantageous in that they when wet, for example when wet with sweat, are unable to give off body heat to a sufficient degree, and therefore wear comfort suffers as a result. This is where the invention provides a remedy through integration of microcapsules 3 comprising phase transition materials in the fibres.

Fibres 2 comprising microcapsules 3 including phase transition materials can be partially mixed with other fibres, or else the fabric can consist of up to 100% of these fibres.

FIG. 5 shows a sanitary napkin as hygiene article 1, the sanitary napkin being formed of a plurality of plies, for example four plies (as depicted), of textile fabrics 1a to 1d.

Since the inner ply or plies facing the wearer of the sanitary napkin is or are closest to the body, at least the inner ply 1d or the two innermost plies 1c and 1d are provided with fibres 2 comprising microcapsules 3. The innermost ply 1d or the two innermost plies 1c and 1d may each be fully formed of fibres 2 comprising microcapsules 3, or else if necessary only partially.

Viscose will be found to be particularly advantageous as a preferred material for at least the ply which is provided with the fibres 2 provided with microcapsules 3, or is formed of these.

It will be appreciated that other materials are also possible instead of viscose and similarly that it is also possible for a plurality of plies of the sanitary napkin to be formed of the fibres 2 provided with the microfibres 3. Similarly, the number of depicted four plies may of course be chosen according to the planned use.

1. Hygiene article, such as sanitary napkin, tampon or incontinence pad, consisting of a textile fabric formed by a multiplicity of fibres, wherein microcapsules (3) having reversible heat-storing properties are included in at least a portion of the fibres (2) or are applied via a coating (4) with a binder atop at least a portion of the fibres (2) or of the surface of the fabric (1), the microcapsules (3) including materials in which a phase transition from solid to liquid, and vice versa, takes place in a temperature range from 15 to 40° C.

2. Hygiene article according to claim 1, wherein microcapsules (3) are endowed with materials in which the phase transition takes place in a temperature range from 18° to 35° C.

3. Hygiene article according to claim 1, wherein a foundation material for the fibres (2) comprises viscose.

4. Hygiene article according to claim 1, 2 or 3, wherein the fibres (2) have been processed to form a fibrous nonwoven web fabric.

5. Hygiene article according to claim 1, wherein the textile fabric (1) consists of a plurality of different fibres (2) and at least one fibre (2) includes the microcapsules (3).

6. Hygiene article according to any one of claims 1 to 5, wherein the proportion of fibres (2) in the textile fabric (1) which are associated with the microcapsules (3) is up to 100%.

7. Hygiene article according to claim 6, wherein there are a plurality of plies (1a,1b,1c,1d) of fabrics (1) and in at least one of the plies (1a,1b,1c,1d) the proportion of fibres (2) which are associated with microcapsules (3) is up to 100%.

8. Hygiene article according to claim 7, wherein at least the inner ply (1d) of the fabrics which faces the wearer of the hygiene article has a proportion of at least approaching 100% of the fibres (2) provided with microcapsules (3).

9. Hygiene article according to claim 7, wherein there are a plurality of plies (1a,1b,1c,1d) of fabrics (1) and in at least the two inner plies (1c,1d) facing the wearer of the hygiene article the proportion of fibres (2) provided with microcapsules (3) is up to 100%.

10. Hygiene article according to claim 7, 8 or 9, wherein at least a portion of the plies of the fabrics (1) of the fibres (2) associated with microcapsules (3) are formed of viscose.

11. Hygiene article according to claim 1, wherein when the microcapsules (3) are applied via a coating (4) atop at least a portion of the surface of the fabric (1) liquid polymer is provided as binder.

12. Hygiene article according to claim 6, wherein the liquid polymer binder consists of acrylates or polyurethane.

13. Hygiene article according to any one of claims 1 to 12, wherein paraffinic hydrocarbons are provided as materials in the microcapsules (3).

14. Hygiene article according to claim 13, wherein octadecane is provided as paraffinic hydrocarbons.

15. Hygiene article according to any one of claims 1 to 14, wherein plastics crystals are provided as materials in the microcapsules (3).

16. Hygiene article according to any one of claims 1 to 15, wherein the microcapsules (3) have a diameter in the range from 0.5μ to 1000μ.

17. Hygiene article according to claim 16, wherein the microcapsules (3) have a diameter in the range from 2 to 30μ.

18. Process for producing a hygiene article, such as a sanitary napkin, a tampon or an incontinence pad, consisting of a textile fabric formed by a multiplicity of fibres, wherein fibrous nonwoven webs are produced from the fibres (2), fibres being used which have reversible heat storage properties and are formed from a foundation material in which a multiplicity of microcapsules (3) are integrated and dispersed, materials in which a phase transition from solid to liquid, and vice versa, takes place in a temperature range from 15 to 40° C. being incorporated in the microcapsules (3).

19. Process according to claim 18, wherein microcapsules (3) are endowed with materials in which the phase transition takes place in a temperature range from 18° to 35° C.

20. Process for producing a hygiene article, such as a sanitary napkin, a tampon or an incontinence pad, consisting of a textile fabric formed by a multiplicity of fibres, wherein at least a portion of the fibres (2) or the surfaces of the textile fabric (1) are provided with a coating (4) with a binder in which microcapsules (3) are dispersed, materials in which a phase transition from solid to liquid, and vice versa, takes place in a temperature range from 15 to 40° C. being incorporated in the microcapsules (3).

21. Process according to claim 20, wherein microcapsules (3) are endowed with materials in which the phase transition takes place in a temperature range from 18° to 35° C.

22. Process according to claim 18 or 20, wherein viscose is used as foundation material for the fibres (2).

23. Process according to any one of claims 18 to 22, wherein paraffinic hydrocarbons are used as materials in the microcapsules (3).

24. Process according to any one of claims 18 to 23, wherein polymer crystals are used as materials in the microcapsules (3).

25. Process according to any one of claims 20 to 24, wherein polyurethanes or acrylates are used as liquid polymer binder.
